(19) United States
(12) Patent Application Publication
(10) Pub. No.: US 2025/0322525 A1
(43) Pub. Date: Oct. 16, 2025

(54) SYSTEMS, METHODS, AND COMPUTER PROGRAMS, FOR ANALYZING IMAGES OF A PORTION OF A PERSON TO DETECT A SEVERITY OF A MEDICAL CONDITION

(71) Applicant: Incyte Corporation, Wilmington, DE (US)

(72) Inventors: Julian Jenkins, Chester Springs, PA (US); Todd Leathers, West Chester, PA (US); Ryad Ali, West Chester, PA (US)

(21) Appl. No.: 19/250,724

(22) Filed: Jun. 26, 2025

Related U.S. Application Data
(62) Division of application No. 17/395,128, filed on Aug. 5, 2021.
(60) Provisional application No. 63/061,572, filed on Aug. 5, 2020.

(51) Int. Cl. G06T 7/00; G06F 18/22; G06T 7/70; G16H 30/40; G16H 50/20; H04N 23/60

(52) U.S. Cl. CPC G06T 7/0016; G06F 18/22; G06T 7/70; G16H 30/40; G16H 50/20; H04N 23/60; G06T 2207/30088; G06T 2207/30096; G06T 2207/30201; G06V 2201/03

(57) ABSTRACT

Methods, systems, and computer programs for monitoring skin condition of a person. In one aspect, a method can include obtaining data representing a first image, the first image depicting skin from at least a portion of a body of a person, generating a severity score that indicates a likelihood that the person is trending towards an increased severity of an auto-immune condition or trending towards a decreased severity of an auto-immune condition, comparing, the severity score to a historical severity score, wherein the historical severity score is indicative of a likelihood that a historical image of the user depicts skin of a person having the auto-immune condition, and determining based on the comparison, whether the person is trending towards an increased severity of the auto-immune condition or trending towards a decreased severity of the auto-immune condition.

### CROSS-REFERENCE TO RELATED APPLICATIONS

This application is a divisional of U.S. application Ser. No. 17/395,128, “SYSTEMS, METHODS, AND COMPUTER PROGRAMS, FOR ANALYZING IMAGES OF A PORTION OF A PERSON TO DETECT A SEVERITY OF A MEDICAL CONDITION,” filed Aug. 5, 2021, which claims the benefit under 35 U.S.C. § 119 (e) of U.S. Patent Application No. 63/061,572, entitled “SYSTEMS, METHODS, AND COMPUTER PROGRAMS, FOR ANALYZING IMAGES OF A PORTION OF A PERSON TO DETECT A SEVERITY OF A MEDICAL CONDITION,” filed Aug. 5, 2020, which is incorporated herein by reference in its entirety.

### BACKGROUND

Vitiligo is a condition that causes the loss of skin color in blotches of skin. This can be caused when pigment-producing cells die or stop functioning.

### SUMMARY

According to one innovative aspect of the present disclosure, a system is disclosed for analyzing an image of a portion of a person's body to determine whether the image depicts a person that is associated with a particular medical condition or a level of change of a severity of a medical condition.

In one aspect, a data processing system for detecting an occurrence of an auto-immune condition is disclosed. The system can include one or more computers, and one or more storage devices storing instructions that, when executed by the one or more computers, cause the one or more computers to perform the operations. In one aspect, the operations can include obtaining, by one or more computers, data representing a first image, the first image depicting skin from at least a portion of a body of a person, providing, by the one or more computers, the data representing the first image as an input to a machine learning model that has been trained to determine a likelihood that image data processed by the machine learning model depicts skin of a person having the auto-immune condition, obtaining, by the one or more computers, output data generated by the machine learning model based on the machine learning model processing the data representing the first image, the output data representing a likelihood that the first image depicts skin of a person having the auto-immune condition, and determining, by the one or more computers, whether the person has the auto-immune condition based on the obtained output data.

Other versions include corresponding devices, methods, and computer programs to perform the actions of methods defined by instructions encoded on computer readable storage devices.

These and other versions may optionally include one or more of the following features. For instance, in some implementations the portion of the body of the person is a face.

In some implementations, obtaining the data representing the first image can include obtaining, by the one or more computers, image data is a selfie image generated by a user device.

In some implementations, obtaining the data representing the first image can include based on a determination that access to a camera of a user device has been granted, obtaining, from time to time, image data representing at least a portion of a body of a person using the camera of the user device, wherein the image data obtained from time to time is image data is generated and obtained without an explicit command from the person to generate and obtain the image data.

According to another innovative aspect of the present disclosure, a data processing system for monitoring skin condition of a person is disclosed. The system can include one or more computers, and one or more storage devices storing instructions that, when executed by the one or more computers, cause the one or more computers to perform the operations. In one aspect, obtaining, by one or more computers, data representing a first image, the first image depicting skin from at least a portion of a body of a person, generating, by the one or more computers, a severity score that indicates a likelihood that the person is trending towards an increased severity of an auto-immune condition or trending towards a decreased severity of an auto-immune condition, wherein generating the severity score includes providing, by the one or more computers, the data representing the first image as an input to a machine learning model that has been trained determine a likelihood that image data processed by the machine learning model depicts skin of a person having the auto-immune condition, and obtaining, by the one or more computers, output data generated by the machine learning model based on the machine learning model processing the data representing the first image, the output data representing a likelihood that the first image depicts skin of a person having the auto-immune condition, wherein the output data generated by machine learning model is the severity score, comparing, by the one or more computers, the severity score to a historical severity score, wherein the historical severity score is indicative of a likelihood that a historical image of the user depicts skin of a person having the auto-immune condition, and determining, by the one or more computers and based on the comparison, whether the person is trending towards an increased severity of the auto-immune condition or trending towards a decreased severity of the auto-immune condition.

Other versions include corresponding devices, methods, and computer programs to perform the actions of methods defined by instructions encoded on computer readable storage devices.

These and other versions may optionally include one or more of the following features. For instance, in some implementations determining whether the person is trending towards an increased severity of the auto-immune condition or trending towards a decreased severity of the auto-immune condition can include determining, by the one or more computers, that the severity score is greater than the historical severity score by more than a threshold amount, and based on determining that the severity score is greater than the historical score by more than a threshold amount, determining that the person is trending towards an increased severity of the auto-immune condition.

In some implementations, determining whether the person is trending towards an increased severity of the auto-immune condition or trending towards a decreased severity of the auto-immune condition can include determining, by the one or more computers, that the severity score is less than the historical severity score by more than a threshold amount, and based on determining that the severity score is less than the historical score by more than a threshold amount, determining that the person is trending towards a decreased severity of the auto-immune condition.

According to another innovative aspect of the present disclosure, a data processing system for detecting an occurrence of a medical condition is disclosed. The system can include one or more computers, and one or more storage devices storing instructions that, when executed by the one or more computers, cause the one or more computers to perform the operations. In one aspect, the operations can include obtaining, by one or more computers, data representing a first image, the first image depicting skin from at least a portion of a body of a person, identifying, by the one or more computers, a historical image that is similar to the first image, determining, by the one or more computers, one or more attributes of the historical image that are to be associated with the first image, generating, by the one or more computers, a vector representation of the first image that includes data describing the one or more attributes, providing, by the one or more computers, the generated vector representation of the first image as an input to the machine learning model that has been trained to determine a likelihood that image data processed by the machine learning model depicts skin of a person having the medical condition, obtaining, by the one or more computers, output data generated by the machine learning model based on the machine learning model processing the generated vectored representation of the first image, and determining, by the one or more computers, whether the person is associated with the medical condition based on the obtained output data.

Other versions include corresponding devices, methods, and computer programs to perform the actions of methods defined by instructions encoded on computer readable storage devices.

These and other versions may optionally include one or more of the following features. For instance, in some implementations the medical condition includes an auto-immune condition.

In some implementations, the one or more attributes include historical image such as lighting conditions, time of day, date, GPS coordinates, facial hair, lesion areas, use of sunblock, use of makeup, or temporary cuts or bruises.

In some implementations, identifying, by the one or more computers, a historical image that is similar to the first image can include determining, by the one or more computers, that the historical image is the most recently stored image of the one or more attributes include data identifying a location of lesion areas in the historical image.

These, and other innovative aspects the present disclosure, are described in more detail in the written description, the drawings, and the claims.

### BRIEF DESCRIPTION OF THE DRAWINGS

FIG. 1 is a diagram of a system for analyzing an image of a portion of a person to determine whether the image depicts a person that is associated with a particular medical condition.

FIG. 2 is a flowchart of a process for analyzing an image of a portion of a person to determine whether the image depicts a person that is associated with a particular medical condition.

FIG. 3 is a flowchart of a process for analyzing an image of a portion of a person to determine whether the image depicts a person that is trending towards an increased severity of a medical condition or trending towards a decreased severity of the particular medical condition.

FIG. 4. is a flowchart of a process for generating an optimized image for input to a machine learning model trained to analyze images of a portion of a person to determine whether the image depicts a person that is associated with a particular medical condition.

FIG. 5 is a diagram of system components that can be used to implement a system for analyzing an image of a portion of a person to determine whether the image depicts a person that is associated with a particular medical condition.

### DETAILED DESCRIPTION

The present disclosure is directed towards systems, methods, and computer programs for analyzing images of persons to detect whether the images depict a person that is associated with a particular medical condition. In some implementations, the particular medical condition can be an autoimmune condition such as vitiligo. Detecting whether a person is associated with a particular medical condition can include detecting that person has the particular medical condition, detecting that the person is trending towards an increased severity of the particular medical condition, detecting that the person is trending towards a decreased severity of the particular medical condition, or detecting that the person does not have the particular medical condition.

Detection of some medical conditions such as medical conditions like vitiligo can require an analysis of variations in the color of pigments, or other aspects, of a person's skin, as depicted by an image of at least a portion of the person's body. Accordingly, such an analysis inherently relies on generation of in input image to an image analysis module that presents the accurate depiction of the patient's skin. A number of environmental factors and non-environmental factors can cause a distortion of an image of a person. For example, environmental factors such as lighting, rain, fog, or the like can cause a distortion in the accurate representation of the pigments of a person's skin in an image. Similarly, non-environmental factors such as camera filters such as a “selfie mode,” “beauty mode,” or programmed image stabilizations or enhancements can cause a distortion in the accurate representation of the pigments of a persons' skin. The present disclosure provides significant technological improvement in that it can preprocess images and modify a vector representation of these images to account for these distortions caused by these environmental factors, non-environmental factors, or both. As a result, vector representations of optimized input images can be generated, for input to an image analysis module of the present disclosure, that more accurately depict pigments of the skin of a person relative to input images generated using conventional systems. Accordingly, determinations as to whether a person depicted by an image is associated with a particular medical condition made, by the present disclosure and based on outputs generated by the image analysis module of the present disclosure, are more accurate than conventional systems.

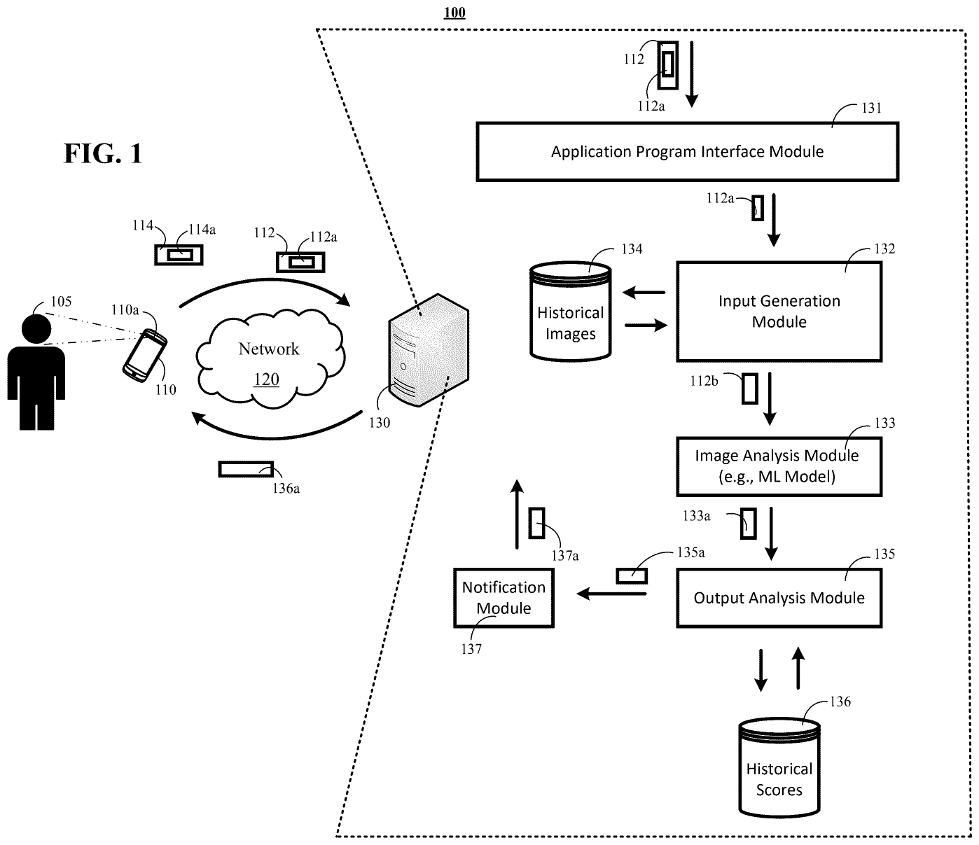

FIG. 1 is a diagram of a system 100 for analyzing an image of a portion of a person to determine whether the image depicts a person that is associated with a particular medical condition. The system 100 can include a user device 110, a network 120, and an application server 130. The application server 130 can include an application programming interface (API) module 131, an input generation module 132, an image analysis module 133, an output analysis module 135, and a notification module 137. The application server 130 can also access images stored in a historical images database 134 and historical scores stored in a historical scores database 136. In some implementations, one or both of these databases can be stored on the application server 130. In other implementations, all, or a portion of, one or more both of these databases may be stored by another computer that is accessible by the application server 130.

For purposes of this specification, the term module can include one or more software components, one or more hardware components, or any combination thereof, that can be used to realize the functionality attributed to a respective module by this specification.

A software component can include, for example, one or more software instructions that, when executed, cause a computer to realize the functionality attributed to a respective module by this specification. A hardware component can include, for example, one or more processors such as a central processing unit (CPU) or graphical processing unit (CPU) that is configured to execute the software instructions to cause the one or more processors to realize the functionality attributed to a module by this specification, a memory device configured to store the software instructions, or a combination thereof. Alternatively, or in addition, a hardware component can include one or more circuits such as a field programmable gate array (FPGA), an application specific integrated circuit (ASIC), or the like, that has been configured to perform operations using hardwired logic to realize the functionality attributed to a module by this specification.

In some implementations, the system 100 can begin performance of a process that generates first image data 112a that represents a first image of a portion of the person's 105 body using a camera 110a of the user device 110. In some implementations, the first image data 112a can include still image data such as a GIF image, a JPEG image, or the like. In some implementations, the first image data 112a can include a video data such as an MPEG-4 video. In some implementations, the user device 110 can include a smartphone. However, in other implementations, the user device 110 can be any device that includes a camera. For example, in some implementations, the user device can be a smartphone, a tablet computer, a laptop computer, a desktop computer, a smartwatch, smartglasses, or the like that includes an integrated camera or is otherwise coupled to a camera. In the example of FIG. 1, the user device 110 uses a camera 110a to capture an image of the person's 105 face. However, the present disclosure is not so limited and instead the camera 110a of the user device 110 can be used to capture an image of any portion of the person's 105 body.

In some implementations, the user device 110 can generate the first image data 112a representing a first image of the portion of the person's 105 body in response to a command of the person 105. For example, the first image data 112a can be generated in response to a user selection of a physical button of the user device 110 or in response to a user selection of a visual representation of a button displayed on a graphical user interface of the user device 110. However, the present disclosure need not be so limited. Instead, in some implementations, the user device 110 can have programmed logic installed on the user device 110 that causes the user device 110 to periodically or asynchronously generate image data of a portion of the person's 105 body.

In the latter scenario, the programmed logic of the user device 110 can configure the user device 110 to detect that a portion of the person's 105 body such as the person's 105 face is within a line of sight of the camera 110a. Then, based on a determination that the portion of the person's body is within a line of sight of the camera 110a, the user device 110 can automatically trigger generation of image data representing an image of the person's 105 face by the user device 110. This ensures that images of the person can be continuously obtained and analyzed regardless of the person's 105 explicit engagement with this system 100. This can be significant in circumstances where the person 105 is potentially associated with a particular medical condition such as vitiligo because the person 105 can be psychologically affected by the changing pigments of their skin and be discouraged from opening an application to take images of themselves for submission to the application server 130 to determine whether a regiment they are on is trending towards an increased severity of vitiligo or trending towards a decreased severity of vitiligo.

The user device 110 can generate a first data structure 112 that includes the first image data 112a and transmit the generated first data structure 112 to the application server 130 using a first data structure 112 using the network 120. The generated first data structure 112 can include fields structuring the first image data 112a and any metadata necessary to transmit the first image data 112a to the application server 130 such as, for example, destination address of the application server 130. In some implementations, the first data structure 112 may implemented as multiple different messages used to transmit the first image data 112a from the user device 110 to the application server 130. For example, the conception first data structure 112 may be implemented by packetizing the image data 112a into multiple different packets and transmitting the packets across the network 120 towards their intended destination of the application server 130. In other implementations, the first data structure 112 may be viewed conceptually as, for example, an electronic message such as an email transmitted via SMTP with the first image data 112a attached to the email. In the example of FIG. 1, the network 120 can include a wired Ethernet network, a wired optical network, a WiFi network, a LAN, a WAN, a cellular network, the Internet, or any combination thereof.

The application server 130 can receive the first data structure 112 via an application programming interface (API) 131. The API 131 can be a software module, hardware module, or a combination thereof that can function as an interface between one or more user devices such as the user device 110 and the application server 131. The API 131 can process the first data structure 112 in order to extract the first image data 112a. The API 131 can provide the first image data 112a as an input to the input generation module 132.

The input generation module 132 can process the first image data 112a to prepare the first image data 112a for input to the image analysis module 133. In some implementations, this may include nominal processing such as vectorising the first image data 112a for input to the image analysis module 133. Vectorizing the first image data 112a can include, for example, generating a vector that includes a plurality of fields, with each field of the vector corresponding to a pixel of the first image data 112a. The generated vector can include a numerical value in each of the vector fields that represents one or more features of the pixel of the image to which the field corresponds. The resulting vector can be a numerical representation of the first image data 112a that is suitable for input and processing by the image analysis module 133. In such implementations, the generated vector can be provided as an input to the image analysis module 133 for further processing by the system 100.

However, in some implementations, such as in the example of FIG. 1, the input generation module 132 can perform additional operations to prepare the first image data 112a for input to the image analysis module 133 prior to providing the first image data 112a as an input to the image analysis module 133. For example, the input generation module 132 can optimize the image 112a for input to the image analysis module 133 based on historical images stored in the historical images database 134 showing portions of the body of the person 105. These historical images stored in the historical images database 134 can include images of the person 105 previously submitted for analysis to the application server 130. In other implementations, the historical images stored in the historical images database 134 can be images obtained from one or more other sources such as images captured during a doctor's visit, images obtained from a social media account associated with the person 105, or the like. These examples of historical images are not to be viewed as limited and historical images of the person 105 stored in the historical images database 134 can be acquired through any means.

In some implementations, one or more of the historical images can be associated with metadata describing attributes of the historical image. For example, metadata can be used to annotate each of a plurality of historical images and provide an indication of attributes of the historical image such as lighting conditions, time of day, date, GPS coordinates, facial hair, lesion areas, use of sunblock, use of makeup, temporary cuts or bruises, or the like. areas tagged as to whether the historical images accurately represent the pigmentation of the person's 105 skin given the environmental factors or non-environmental factors associated with the historical image. In some implementations, these tags can be assigned by a human user based on a review of historical images.

The input generation module 132 can optimize the image 112a using the historical images stored in the historical images database 134 in a number of different ways. For purposes of the present disclosure, “optimizing” an image such as image 112a can include generating data that (i) represents the image or (ii) is associated with the image that can be provided as an input to the image analysis module 133 in order to make the image 112a better suited for processing by the image analysis module 133. An optimized image can be better suited for processing by the image analysis module if the optimized image causes the image analysis module 133 to generate better output data 133a than the image analysis module 133 would have generated had the image analysis module 133 processed the image priority to its optimization. A better output can include, for example, output that causes the output analysis module 135 to make more accurate determinations, based on the output data 133a generated by the image analysis module 133, as to whether the person is associated with a particular medical condition, is trending towards an increased severity of the particular medical condition, is trending towards a decreased severity of the particular medical condition, or is not associated with the particular medical condition.

In some implementations, an image 112a can be processed by the input generation module 132 to generate an optimized image 112b in a number of different ways. In one implementation, the input generation module can perform a comparison of a newly received image 112a to historical images 134. Upon identifying historical images that are sufficiently similar to the optimized image 112b, the input generation module 132 can set values of one or more fields of an image vector that correspond to metadata attributes of the identified historical images that were determined to be similar to the input image 112a.

For example, the input generation module 132 can determine that the newly obtained image 112a is similar to one of the historical images. In some implementations, similarity may be determined based on image similarity based on, for example, a vector-based comparison of a vector representing the image 112a and one or more vectors representing respective historical images. Upon determining that a newly obtained image 112a is similar to a historical image captured in particular lighting conditions, the input generation module 132 can set a field of an image vector representation of the optimized image 112b indicating that the image 112a was taken during particular lighting conditions. This additional information can provide a signal to the image analysis module 132 that can inform inferences made by the image analysis module 133.

By way of another example, upon determining that a newly obtained image 112a is similar to a historical image captured with the person 105 wearing sunblock, the input generation module 132 can set a field of an image vector representation of the optimized image 112b indicating that the image 112a was taken with the person 105 depicted in the image wearing sunblock. This additional information can provide a signal to the image analysis module 133 that can inform inferences made by the image analysis module 133.

By way of another example, the input generation modules can determine a relationship between a newly obtained image 112a and a similar historical image. In some implementations, similarity between the image 112a and a historical image can be determined based on a temporal relationship between the images. For example, a particular historical image may be determined to be similar to the image 112a if the historical image is the most recently captured or stored image depicting a portion of the person's 105 skin. In such instances, the input generation module 133 can generate data for inclusion in the vector 112b representing the optimized image based on metadata associated with the similar historical image indicating a location of a previously known vitiligo lesion depicted on the skin of the person 105 depicted by the historical image. This additional information can provide a signal to the image analysis module 133 that can inform inferences made by the image analysis module 133.

Nothing in these examples should be interpreted as limiting the scope of the present disclosure. Instead, the any metadata describing any attribute of any historical photo can be used to optimize an image for input to an image analysis module 133.

The input generation module 132 can generate a vector representation of the optimized image 112b for input to the image analysis module. The vector representation can include a vector that includes a plurality of fields, with each field of the vector corresponding to a pixel of the first image data 112a and one or more fields representing additional information attributed to the first image data 112a from one or more similar historical images. The generated vector 1112b can include a numerical value in each of the vector fields that represents one or more features of the pixel of the image to which the field corresponds and one or more numerical values indicating the presence, absence, degree, location, or other feature of the additional information attributed to the input image.

The image analysis module 133 can be configured to analyze the vector representation of the optimized image 112b and generate output data 133a indicating a likelihood that the image 112a represented by the vector representation of the optimized image 112b depicts a person associated with a medical condition such as vitiligo. The output data 133a generated by the image analysis model 133 based on the image analysis module 133 processing the vector representing the optimized image data 112b can be analyzed by an output analysis module 135 to determine whether the person 105 is associated with the medical condition.

In some implementations, the image analysis module 133 can include one or more machine learning models that have been trained to determine a likelihood that image data such as a vector representation of the optimized image data 112b processed by the machine learning model represents an image depicting skin of a person 105 having a medical condition such as one or more auto-immune conditions. In some implementations, the auto-immune conditions can be vitiligo. That is, the machine learning model can be trained to generate a output data 133a that may represent a value such as a probability that the person depicted by the image data represented by the vector representation 112b processed by the machine learning model is a person that likely has vitiligo or the person that likely does not have vitiligo. However, the machine learning model does not, by itself, actually classify the output data 133a generated by the machine learning model. Instead, the machine learning model generates the output data 133a and provides the output data 133a to the output analysis module 135 that can be configured to threshold the output data 133a into one or classes of persons 105.

The machine learning model can be trained in a number of different ways. In one implementation, training can be achieved using a simulator to generate training labels for training vectors representing optimized images. The training labels can provide an indication as to whether the training vector representation corresponds to an image of a person that is associated with a medical condition or an image of a person that is not associated with a medical condition. In such implementations, each training vector representing an optimized image can be provided as an input to the machine learning model, processed by the machine learning model, and then training output generated by the machine learning model can be used to determine a predicted label for the training vector representation. The predicted label for training vector representation can be compared to the training label corresponding to the processed training vector representation. Then, the parameters of the first machine learning model can be adjusted based on differences between the predicted label and the training label. This process can iteratively continue for each of a plurality of training vectors representations until the predicted labels for a newly processed training vector representation begin to match, within a predetermined level of error, a training label generated by the simulator for the training vector representation.

The output data 133a generated by the image analysis unit 133 such as a machine learning model that has been trained to process a vector representation of an optimized image and generate the output data 133a indicate of a likelihood that the image corresponding to the vector representation depicts a person associated with a particular medical condition can be provided as an input to the output analysis module 135. The output analysis module 135 can receive the output analysis module 135 apply one or more business logic rules to the output data 133a such as a probability to determine whether or not the person that was depicted in the image 112a upon which the vector representation of the optimized image was based is associated with a medical condition or not associated with a medical condition.

In such in implementation, a single threshold can be used, by the output analysis module 135 to evaluate the output data 133a. For example, in some implementations, the output analysis module 135 can obtain the output data 133a such as a probability and compare the obtained output data 133a to a predetermined threshold. If the output analysis module 135 determines that the obtained output data 133a does not satisfy the predetermined threshold, then the output analysis module 135 can determine that the person 105 is not associated with a particular medical condition. Alternatively, if the output analysis module 135 determines that the obtained output data 133a satisfies the predetermined threshold, then the output analysis module 135 can determine that the person 105 is associated with the particular medical condition.

In some implementations, the output analysis module 135 can generate output data 135a that includes data indicating the determination made, by the output analysis module 135 and based on the generated output data 133a, regarding whether the person 105 is associated with the medical condition. The notification module 137 can generate a notification 137a that includes rendering that, when rendered by the user device 110, causes the user device to display an alert or other visual message on the display of the user device 110 that communicates, to the person 105, the determination made by the output analysis module 135. However, the present disclosure need not be so limited. For example, the notification 137a may be configured to communicate the determination of the output analysis module 135 in other ways when it is processed by the user device 110. For example, the notification 137a may be configured to, when processed by the user device 110, cause haptic feedback or an audio message separate from or in combination with the visual message to convey the results of the determination of the output analysis module 135 based on the output data 133a. The notification 137a can be transmitted, by the application server 130, to the user device 110 via the network 120.

However, the subject matter of this specification is not limited to the application server 130 transmitting the notification 137a to the user device 110. For example, the application server 130 can also transmit the notification 137a to another computer such as a different user device. In some implementations, for example, the notification 137a can be transmitted to a user device of the person's 105 doctor, family member, or other person.

The output analysis module 135 is also capable of making other types of determinations. In some implementations, for example, the output analysis module 135 can make determinations as to whether a vector representation of an optimized image corresponds to an image that depicts a person that is trending towards an increased severity of a medical condition or trending towards a decreased severity of the medical condition.

By way of example and with reference to FIG. 1, the output analysis module 135 can store the output data 133a such as a probability or severity score in the historical scores 136 database after the image analysis module 133 generates the output data based on processing of the vector representation of an optimized image 112b. This output data can used as a severity score that represents a level of severity of the medical condition associated with the patient 105 depicted by the image 112a. In some implementations, this severity score can indicate a likelihood that the person 105 is trending towards an increased severity of a medical condition or trending towards a decreased severity of the medical condition. Then, at a subsequent point in time, the user device 110 can use the camera 110a to capture a second image 114a of the user 105. The user device 110 can use a second data structure 114 to transmit the second image 114a to the application server via the network 120. The API module 131 can receive the second data structure, extract the image 114a, and then provide the image 114a as an input to the input generation module 132.

Continuing with this example, the input generation module 132 can perform the operations described above to optimize the image 114a. In some implementations, this can include performing searches of the historical image database 134 and porting attributes of one or more historical images to the current image 114a. The input generation module 132 can generate a second vector representation of the optimized image 114b based on the ported attributes. The input generation module 132 can provide the second vector representation of the optimized image 114b as an input to the image analysis module 133. The image analysis module 133 can process the second vector representation of the optimized image 114b and generate second output data 133b, which indicates a likelihood that the second image 114a depicts a person 105 that is associated with a particular medical condition.

At this point, the output analysis module 135 can analyze the second output data 133b generated based on the second vector representation of the optimized image 114b in view of the first output data 133a generated based on the first vector representation of the optimized image 112b. In particular, the output analysis module 135 can determine whether the person 105 depicted by the image 114a is trending towards an increased severity of a particular medical condition or trending towards a decreased severity of the particular medical condition based on the change of the second output data 133b relative to the first output data 133. For example, assume that a scale is establish where an output value of “1” means the person has the medical condition and an output value of “0” means that the person does not have the medical condition. Under a scale like this, if the first output data 133a was 0.65 and the second output data 133b was. 78, the difference between the first output data 133a and the second output data 133b indicates that the person 105 is trending towards an increased severity of the medical condition. Likewise, under the same scale and a scenario where the first output data 133a is 0.65 and the second output data 133b was 0.49, the difference between the first output data 133a and the second output data 133b indicates that the person 105 is trending towards a decreased severity of the medical condition.

None of these examples limit the present disclosure. For example, other scales can be used such as “1” meaning that a person does not have the medical condition and “0” means the person has the medical condition. By way of another example, a scale can be determined that has “−1” meaning that a person does not have the medical condition and a “1” meaning that a person does have the medical condition. Indeed, any scale may be used and can be adjusted based on the range of output data 133a, 133b values generated by the image generation module 132.

However, the present disclosure need not be so limited. For example, in some implementations, the output analysis module 135 can use other processes, systems, or a combination thereof, to determine whether a person depicted by an image 114a is trending towards an increased severity of a particular medical condition or trending towards a decreased severity of the particular medical condition. For example, in some implementations, the output analysis module 135 can be comprised of one or more machine learning models that are trained to predict whether output data 133a produced by the ML Model 133 indicates that the person depicted by the image 114a is trending towards an increased severity of a particular medical condition or trending towards a decreased severity of the particular medical condition.

In more detail, the output analysis module 135 of such an implementation can include one or more machine learning models that have been trained to determine a likelihood that a person associated with a current severity score generated based on image 114a and one or more historical severity scores such as the severity score generated based on image 112a is trending towards an increased severity of an medical (e.g., auto-immune) condition or trending towards a decreased severity of a medical (e.g., auto-immune) condition. That is, the machine learning model can be trained to generate a output data 135a that may represent a value such as a probability that the person associated with a current severity score generated based on image 114a and one or more historical severity scores such as the severity score generated based on image 112a is trending towards an increased severity of an medical (e.g., auto-immune) condition or trending towards a decreased severity of a medical (e.g., auto-immune) condition. Then, the output data produced by the one or more machine learning models of the output analysis module 135 can be analyzed to determine whether the person associated with the current severity score and the one or more historical severity scores is trending towards an increased severity of an medical (e.g., auto-immune) condition or trending towards a decreased severity of a medical (e.g., auto-immune) condition. In some implementations, the one or more machine learning models can be trained to receive, as inputs, multiple historical severity scores in addition to the current severity score in order to provide more data signals that the machine learning model can consider in determining whether the person associated with the severity scores is trending towards or aware from the medical condition.

Decisions made by the output analysis unit 135 can be transmitted to the user device 110 or other user device using the notification module 137a. For example, the output analysis module 135 can output data 135a indicating whether the person 105 is trending towards an increased severity of the medical condition, trending towards a decreased severity of the medical condition, no change in the severity of the medical condition, or the like. The output data 135a can be provided to the notification module 137 and the notification module can generation a notification 137a based on the output data 135. The application server 130 can notify the user device 110 or other user device by transmitting the notification 137a to one or more of the respective user devices.

Additional applications can be used to analyze the output data 135a indicating whether the person 105 is trending towards an increased severity of the medical condition, trending towards a decreased severity of the medical condition, or no change in the severity of the medical condition. In some implementations, for example, output data 135a or the notification 137a can include data represent the degree of the change between a first output data 133a and second output data 133b based on the vectors corresponding to the first image data 112a and second image data 114a, respectively. Software on the user device 110 or another user device can analyze the degree of change between the first output data 133a and second output data 133b and generate one or more alerts to the person 105 or the persons doctor. Such alerts can remind the person 105 to apply his/her medicine, suggest that a doctor adjust the person's prescription, or the like. For example, in some implementations such as where the medical condition is vitiligo, the software can be configured to determine that the difference between the first output data 133a and the second output data 133b indicates that the user is trending towards more severe vitiligo lesions. In such instances, the software can generate alerts reminding the person 105 to apply his/her medicine, suggest that the person 105 apply his/her medicine more often, or suggest to a doctor to increase a dosage of the person's 105 medicine based on the degree of the change between the first output data 133a and the second output data 133b. Other applications of similar scope are also intended to fall within the scope of the present disclosure. Though the analysis for these reminder alerts/suggestion alerts are described as being performed by applications on user devices, the present disclosure is not so limited. Instead, the analysis of the degree of difference between output data 133a and output data 133b can be performed by the output analysis module 135 on the application server 130 and the reminder alerts/suggestion alerts can be generated by the notification module 137.

Though the notification module 137 is not explicitly shown has passing the notification 137a through the API module 131, it considered that, in some implementations, data communications between a user device and the application server occur via the API 131 as a form of middleware between the application server 130 and the user device(s).

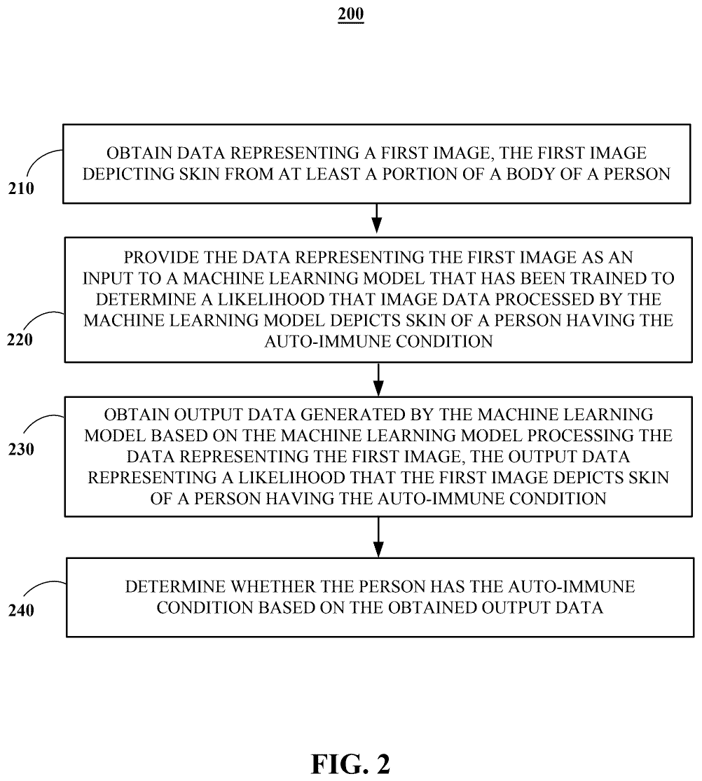

FIG. 2 is a flowchart of a process 200 for analyzing an image of a portion of a person to determine whether the image depicts a person that is associated with a particular medical condition. In general, the process 200 can include obtaining, by one or more computers, data representing a first image, the first image depicting skin from at least a portion of a body of a person (210), providing, by the one or more computers, the data representing the first image as an input to a machine learning model that has been trained to determine a likelihood that image data processed by the machine learning model depicts skin of a person having the auto-immune condition (220), obtaining, by the one or more computers, output data generated by the machine learning model based on the machine learning model processing the data representing the first image, the output data representing a likelihood that the first image depicts skin of a person having the auto-immune condition (230), and determining, by the one or more computers, whether the person has the auto-immune condition based on the obtained output data (240).

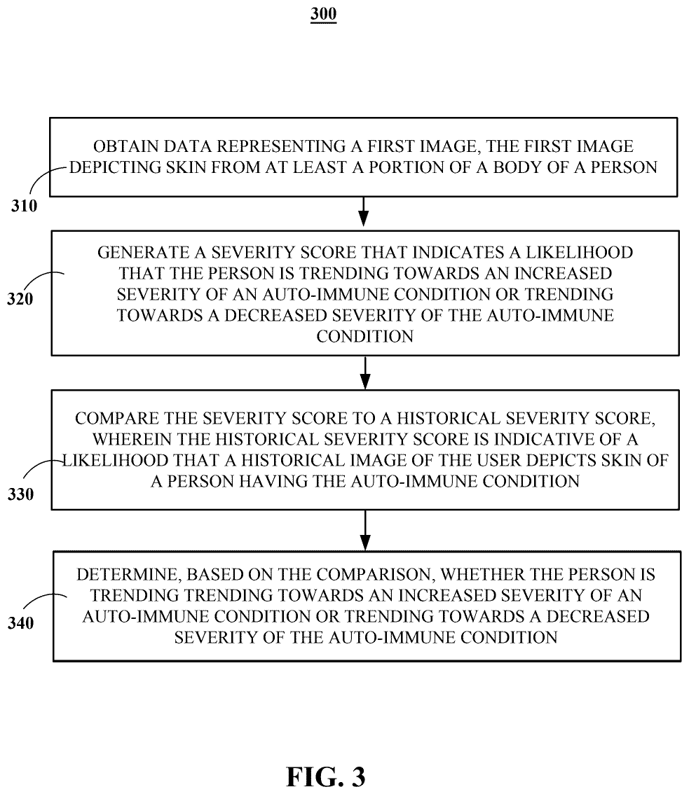

FIG. 3 is a flowchart of a process 300 for analyzing an image of a portion of a person to determine whether the image depicts a person that is trending towards an increased severity of a medical condition or trending towards a decreased severity of the particular medical condition. For example, in some implementations, the process 300 can include obtaining, by one or more computers, data representing a first image, the first image depicting skin from at least a portion of a body of a person (310), generating, by the one or more computers, a severity score that indicates a likelihood that the person is trending towards an increased severity of an auto-immune condition or trending towards a decreased severity of the auto-immune condition (320), comparing, by the one or more computers, the severity score to a historical severity score, wherein the historical severity score is indicative of a likelihood that a historical image of the user depicts skin of a person having the auto-immune condition (330), and determining, by the one or more computers and based on the comparison, whether the person is trending towards an increased severity of an auto-immune condition or trending towards an increased severity of the auto-immune condition (340).

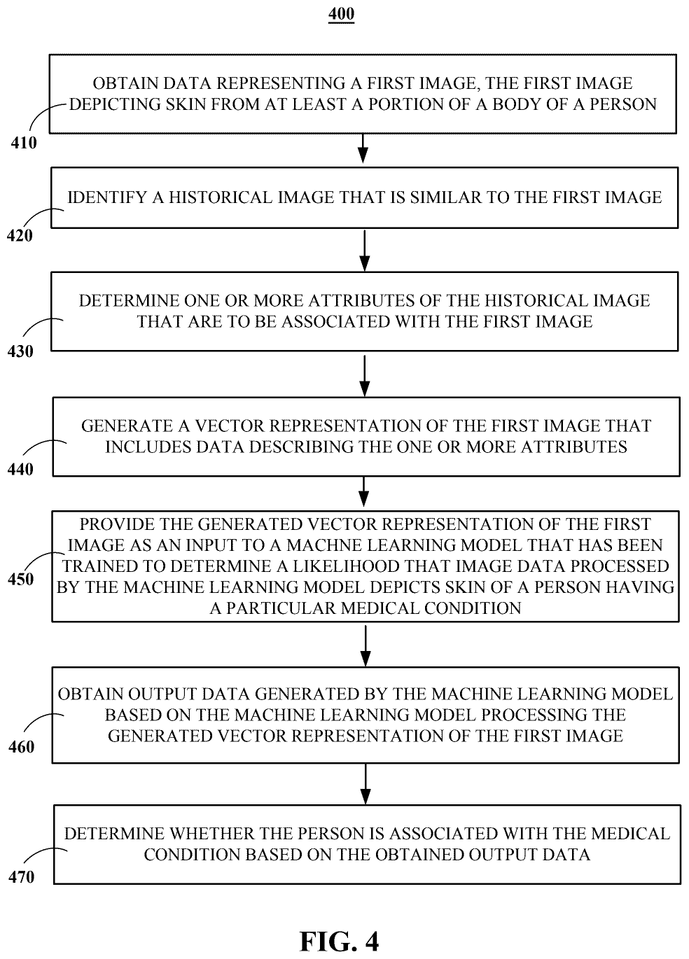

FIG. 4. is a flowchart of a process 400 for generating an optimized image for input to a machine learning model trained to analyze images of a portion of a person to determine whether the image depicts a person that is associated with a particular medical condition. In general, the process 400 can include obtaining, by one or more computers, data representing a first image, the first image depicting skin from at least a portion of a body of a person (410), identifying, by the one or more computers, a historical image that is similar to the first image (420), determining, by the one or more computers, one or more attributes of the historical image that are to be associated with the first image (430), generating, by the one or more computers, a vector representation of the first image that includes data describing the one or more attributes (440), providing, by the one or more computers, the generated vector representation of the first image as an input to the machine learning model that has been trained to determine a likelihood that image data processed by the machine learning model depicts skin of a person having a particular medical condition (450), obtaining, by the one or more computers, output data generated by the machine learning model based on the machine learning model processing the generated vectored representation of the first image (460), and determining, by the one or more computers, whether the person is associated with the medical condition based on the obtained output data (470).

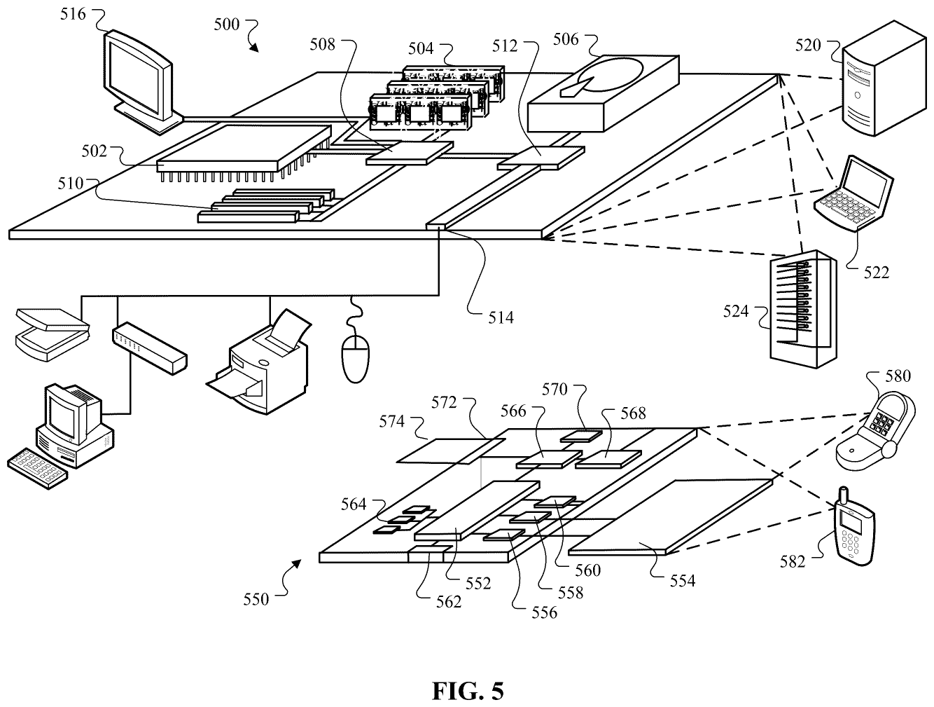

FIG. 5 is a diagram of system components that can be used to implement a system for analyzing an image of a portion of a person to determine whether the image depicts a person that is associated with a particular medical condition.

Computing device 500 is intended to represent various forms of digital computers, such as laptops, desktops, workstations, personal digital assistants, servers, blade servers, mainframes, and other appropriate computers. Computing device 550 is intended to represent various forms of mobile devices, such as personal digital assistants, cellular telephones, smartphones, and other similar computing devices. Additionally, computing device 500 or 550 can include Universal Serial Bus (USB) flash drives. The USB flash drives can store operating systems and other applications. The USB flash drives can include input/output components, such as a wireless transmitter or USB connector that can be inserted into a USB port of another computing device. The components shown here, their connections and relationships, and their functions, are meant to be exemplary only, and are not meant to limit implementations of the inventions described and/or claimed in this document.

Computing device 500 includes a processor 502, memory 504, a storage device 506, a high-speed interface 508 connecting to memory 504 and high-speed expansion ports 510, and a low speed interface 512 connecting to low speed bus 514 and storage device 506. Each of the components 502, 504, 506, 508, 510, and 512, are interconnected using various busses, and can be mounted on a common motherboard or in other manners as appropriate. The processor 502 can process instructions for execution within the computing device 500, including instructions stored in the memory 504 or on the storage device 506 to display graphical information for a GUI on an external input/output device, such as display 516 coupled to high speed interface 508. In other implementations, multiple processors and/or multiple buses can be used, as appropriate, along with multiple memories and types of memory. Also, multiple computing devices 500 can be connected, with each device providing portions of the necessary operations, e.g., as a server bank, a group of blade servers, or a multi-processor system.

The memory 504 stores information within the computing device 500. In one implementation, the memory 504 is a volatile memory unit or units. In another implementation, the memory 504 is a non-volatile memory unit or units. The memory 504 can also be another form of computer-readable medium, such as a magnetic or optical disk.

The storage device 506 is capable of providing mass storage for the computing device 500. In one implementation, the storage device 506 can be or contain a computer-readable medium, such as a floppy disk device, a hard disk device, an optical disk device, or a tape device, a flash memory or other similar solid state memory device, or an array of devices, including devices in a storage area network or other configurations. A computer program product can be tangibly embodied in an information carrier. The computer program product can also contain instructions that, when executed, perform one or more methods, such as those described above. The information carrier is a computer- or machine-readable medium, such as the memory 504, the storage device 506, or memory on processor 502.

The high speed controller 508 manages bandwidth-intensive operations for the computing device 500, while the low speed controller 512 manages lower bandwidth intensive operations. Such allocation of functions is exemplary only. In one implementation, the high-speed controller 508 is coupled to memory 504, display 516, e.g., through a graphics processor or accelerator, and to high-speed expansion ports 510, which can accept various expansion cards (not shown). In the implementation, low-speed controller 512 is coupled to storage device 506 and low-speed expansion port 514. The low-speed expansion port, which can include various communication ports, e.g., USB, Bluetooth, Ethernet, wireless Ethernet can be coupled to one or more input/output devices, such as a keyboard, a pointing device, microphone/speaker pair, a scanner, or a networking device such as a switch or router, e.g., through a network adapter. The computing device 500 can be implemented in a number of different forms, as shown in the figure. For example, it can be implemented as a standard server 520, or multiple times in a group of such servers. It can also be implemented as part of a rack server system 524. In addition, it can be implemented in a personal computer such as a laptop computer 522. Alternatively, components from computing device 500 can be combined with other components in a mobile device (not shown), such as device 550. Each of such devices can contain one or more of computing device 500, 550, and an entire system can be made up of multiple computing devices 500, 550 communicating with each other.

The computing device 500 can be implemented in a number of different forms, as shown in the figure. For example, it can be implemented as a standard server 520, or multiple times in a group of such servers. It can also be implemented as part of a rack server system 524. In addition, it can be implemented in a personal computer such as a laptop computer 522. Alternatively, components from computing device 500 can be combined with other components in a mobile device (not shown), such as device 550. Each of such devices can contain one or more of computing device 500, 550, and an entire system can be made up of multiple computing devices 500, 550 communicating with each other.

Computing device 550 includes a processor 552, memory 564, and an input/output device such as a display 554, a communication interface 566, and a transceiver 568, among other components. The device 550 can also be provided with a storage device, such as a micro-drive or other device, to provide additional storage. Each of the components 550, 552, 564, 554, 566, and 568, are interconnected using various buses, and several of the components can be mounted on a common motherboard or in other manners as appropriate.

The processor 552 can execute instructions within the computing device 550, including instructions stored in the memory 564. The processor can be implemented as a chipset of chips that include separate and multiple analog and digital processors. Additionally, the processor can be implemented using any of a number of architectures. For example, the processor 510 can be a CISC (Complex Instruction Set Computers) processor, a RISC (Reduced Instruction Set Computer) processor, or a MISC (Minimal Instruction Set Computer) processor. The processor can provide, for example, for coordination of the other components of the device 550, such as control of user interfaces, applications run by device 550, and wireless communication by device 550.

Processor 552 can communicate with a user through control interface 558 and display interface 556 coupled to a display 554. The display 554 can be, for example, a TFT (Thin-Film-Transistor Liquid Crystal Display) display or an OLED (Organic Light Emitting Diode) display, or other appropriate display technology. The display interface 556 can comprise appropriate circuitry for driving the display 554 to present graphical and other information to a user. The control interface 558 can receive commands from a user and convert them for submission to the processor 552. In addition, an external interface 562 can be provide in communication with processor 552, so as to enable near area communication of device 550 with other devices. External interface 562 can provide, for example, for wired communication in some implementations, or for wireless communication in other implementations, and multiple interfaces can also be used.

The memory 564 stores information within the computing device 550. The memory 564 can be implemented as one or more of a computer-readable medium or media, a volatile memory unit or units, or a non-volatile memory unit or units. Expansion memory 574 can also be provided and connected to device 550 through expansion interface 572, which can include, for example, a SIMM (Single In Line Memory Module) card interface. Such expansion memory 574 can provide extra storage space for device 550, or can also store applications or other information for device 550. Specifically, expansion memory 574 can include instructions to carry out or supplement the processes described above, and can include secure information also. Thus, for example, expansion memory 574 can be provide as a security module for device 550, and can be programmed with instructions that permit secure use of device 550. In addition, secure applications can be provided via the SIMM cards, along with additional information, such as placing identifying information on the SIMM card in a non-hackable manner.

The memory can include, for example, flash memory and/or NVRAM memory, as discussed below. In one implementation, a computer program product is tangibly embodied in an information carrier. The computer program product contains instructions that, when executed, perform one or more methods, such as those described above. The information carrier is a computer- or machine-readable medium, such as the memory 564, expansion memory 574, or memory on processor 552 that can be received, for example, over transceiver 568 or external interface 562.

Device 550 can communicate wirelessly through communication interface 566, which can include digital signal processing circuitry where necessary. Communication interface 566 can provide for communications under various modes or protocols, such as GSM voice calls, SMS, EMS, or MMS messaging, CDMA, TDMA, PDC, WCDMA, CDMA2000, or GPRS, among others. Such communication can occur, for example, through radio-frequency transceiver 568. In addition, short-range communication can occur, such as using a Bluetooth, Wi-Fi, or other such transceiver (not shown). In addition, GPS (Global Positioning System) receiver module 570 can provide additional navigation- and location-related wireless data to device 550, which can be used as appropriate by applications running on device 550.

Device 550 can also communicate audibly using audio codec 560, which can receive spoken information from a user and convert it to usable digital information. Audio codec 560 can likewise generate audible sound for a user, such as through a speaker, e.g., in a handset of device 550. Such sound can include sound from voice telephone calls, can include recorded sound, e.g., voice messages, music files, etc. and can also include sound generated by applications operating on device 550.

The computing device 550 can be implemented in a number of different forms, as shown in the figure. For example, it can be implemented as a cellular telephone 580. It can also be implemented as part of a smartphone 582, personal digital assistant, or other similar mobile device.

Various implementations of the systems and methods described here can be realized in digital electronic circuitry, integrated circuitry, specially designed ASICs (application specific integrated circuits), computer hardware, firmware, software, and/or combinations of such implementations. These various implementations can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms “machine-readable medium” “computer-readable medium” refers to any computer program product, apparatus and/or device, e.g., magnetic discs, optical disks, memory, Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor.

To provide for interaction with a user, the systems and techniques described here can be implemented on a computer having a display device, e.g., a CRT (cathode ray tube) or LCD (liquid crystal display) monitor for displaying information to the user and a keyboard and a pointing device, e.g., a mouse or a trackball by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input.

The systems and techniques described here can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the systems and techniques described here, or any combination of such back end, middleware, or front end components. The components of the system can be interconnected by any form or medium of digital data communication, e.g., a communication network. Examples of communication networks include a local area network (“LAN”), a wide area network (“WAN”), and the Internet.

The computing system can include clients and servers. A client and server are generally remote from each other and typically interact through a communication network. The relationship of client and server arises by virtue of computer programs running on the respective computers and having a client-server relationship to each other.

### OTHER EMBODIMENTS

A number of embodiments have been described. Nevertheless, it will be understood that various modifications can be made without departing from the spirit and scope of the invention. In addition, the logic flows depicted in the figures do not require the particular order shown, or sequential order, to achieve desirable results. In addition, other steps can be provided, or steps can be eliminated, from the described flows, and other components can be added to, or removed from, the described systems. Accordingly, other embodiments are within the scope of the following claims.

1. A method for detecting an occurrence of a medical condition, the method comprising:
obtaining, by one or more computers, data representing a first image, the first image depicting skin from at least a portion of a body of a person;
generating, by one or more computers, a vector representation of the first image, the vector representation including a plurality of fields, each field corresponding to a pixel of the first image;
identifying, by the one or more computers, a historical image that is similar to the first image;
determining, by the one or more computers, one or more attributes of the historical image that are to be associated with the first image, wherein the one or more attributes describe a distortion of the historical image;
modifying, by the one or more computers, the vector representation to specify that the determined one or more attributes describe a distortion of the skin depicted in the first image by:
modifying, by the one or more computers, the vector representation of the first image that to include one or more other fields describing the one or more attributes o the historical image that is similar to the first image;
providing, by the one or more computers, the modified vector representation of the first image as an input to the machine learning model that has been trained to determine a likelihood that first image data depicts skin of a person having the medical condition;
obtaining, by the one or more computers, output data generated by the machine learning model based on the machine learning model processing the modified vector representation of the first image; and
determining, by the one or more computers, whether the person is associated with the medical condition based on the obtained output data.

2. The method of claim 1, wherein the medical condition includes an auto-immune condition.

3. The method of claim 1, wherein the one or more attributes include one or more attributes of the historical image such as lighting conditions, time of day, date, GPS coordinates, facial hair, lesion areas, use of sunblock, use of makeup, or temporary cuts or bruises.

4. The method of claim 1, wherein identifying, by the one or more computers, a historical image that is similar to the first image comprises:
determining, by the one or more computers, that the historical image is the most recently stored image of the person.

5. The method of claim 4, wherein the one or more attributes include data identifying a location of lesion areas in the historical image.

6. A data processing system for detecting an occurrence of a medical condition, comprising:
one or more computers; and
one or more storage devices storing instructions that, when executed by the one or more computers, cause the one or more computers to perform the operations comprising:
obtaining, by one or more computers, data representing a first image, the first image depicting skin from at least a portion of a body of a person;
generating, by one or more computers, a vector representation of the first image, the vector representation including a plurality of fields, each field corresponding to a pixel of the first image;
identifying, by the one or more computers, a historical image that is similar to the first image;
determining, by the one or more computers, one or more attributes of the historical image that are to be associated with the first image, wherein the one or more attributes describe a distortion of the historical image;
modifying, by the one or more computers, the vector representation to specify that the determined one or more attributes describe a distortion of the skin depicted in the first image by:
modifying, by the one or more computers, the vector representation of the first image to include one or more other fields describing the one or more attributes; providing, by the one or more computers, the modified vector representation of the first image as an input to the machine learning model that has been trained to determine a likelihood that first image data depicts skin of a person having the medical condition;
obtaining, by the one or more computers, output data generated by the machine learning model based on the machine learning model processing the modified vector representation of the first image; and
determining, by the one or more computers, whether the person is associated with the medical condition based on the obtained output data.

7. The system of claim 6, wherein the medical condition includes an auto-immune condition.

8. The system of claim 6, wherein the one or more attributes include one or more attributes of the historical image such as lighting conditions, time of day, date, GPS coordinates, facial hair, lesion areas, use of sunblock, use of makeup, or temporary cuts or bruises.

9. The system of claim 6, wherein identifying, by the one or more computers, a historical image that is similar to the first image comprises:
determining, by the one or more computers, that the historical image is the most recently stored image of the person.

10. The system of claim 9, wherein the one or more attributes include data identifying a location of lesion areas in the historical image.

11. A non-transitory computer-readable medium storing software comprising instructions executable by one or more computers which, upon such execution, cause the one or more computers to perform the operations comprising:
obtaining, by one or more computers, data representing a first image, the first image depicting skin from at least a portion of a body of a person;
identifying, by the one or more computers, a historical image that is similar to the first image;
determining, by the one or more computers, one or more attributes of the historical image that are to be associated with the first image;
generating, by the one or more computers, a vector representation of the first image that includes data describing the one or more attributes;
providing, by the one or more computers, the generated vector representation of the first image as an input to the machine learning model that has been trained to determine a likelihood that image data processed by the machine learning model depicts skin of a person having the medical condition;
obtaining, by the one or more computers, output data generated by the machine learning model based on the machine learning model processing the generated vectored representation of the first image; and
determining, by the one or more computers, whether the person is associated with the medical condition based on the obtained output data.

12. The computer-readable medium of claim 11, wherein the medical condition includes an auto-immune condition.

13. The computer-readable medium of claim 11, wherein the one or more attributes include historical image such as lighting conditions, time of day, date, GPS coordinates, facial hair, lesion areas, use of sunblock, use of makeup, or temporary cuts or bruises.

14. The computer-readable medium of claim 11, wherein identifying, by the one or more computers, a historical image that is similar to the first image comprises:
determining, by the one or more computers, that the historical image is the most recently stored image of the person.

15. The computer-readable medium of claim 14, wherein the one or more attributes include data identifying a location of lesion areas in the historical image.
